(19) United States
(12) Patent Application Publication
(10) Pub. No.: US 2011/0152937 A1
(43) Pub. Date: Jun. 23, 2011

(54) Surgical Implants for Selectively Controlling Spinal Motion Segments

(75) Inventor: Hai H. Trieu, Cordova, TN (US)

(73) Assignee: WARSAW ORTHOPEDIC, INC., Warsaw, IN (US)

(21) Appl. No.: 12/645,053

(22) Filed: Dec. 22, 2009

(51) Int. Cl. A61B 17/70; A61B 17/88

(52) U.S. Cl. 606/264; 606/279

(57) ABSTRACT

Elongated connecting elements include bodies having composite cross-sections defining a first resistance to bending about a first bending axis and a second resistance to bending about a second bending axis that is transverse to the first bending axis. The cross-section also includes intermediate bending axes between the first and second bending axes that provide resistance to bending that is less than that provided about the first bending axis and greater than that provided about the second bending axis. The connecting elements are positioned along one or more spinal motion segments and engaged to vertebrae with anchors with one of the first, second, and intermediate bending axes in the desired orientation relative to the spinal motion segment to provide the desired stiffness and resistance to bending.

### BACKGROUND

Various devices and methods for stabilizing bone structures have been used for many years. For example, one type of stabilization technique uses one or more elongated rods extending between components of a bony structure and secured to the bony structure to stabilize the components relative to one another. The components of the bony structure are exposed and one or more bone engaging fasteners are placed into each component. The elongated rod is then secured to the bone engaging fasteners in order to stabilize the components of the bony structure.

One problem associated with the above described stabilization structures is that the stabilization structure can provide the same stabilization effect in all planes of motion of a spinal motion segment. Other systems provide elongated rods that provide differing resistance to bending about various axes, but are not readily engageable to the bone engaging fasteners in all orientations of the rod relative to the bone engaging fasteners due to the outer dimensions of the rod varying about longitudinal axis of the rod.

### SUMMARY

Elongated connecting elements include bodies having composite cross-sections defining a first resistance to bending about a first bending axis and a second resistance to bending about a second bending axis that is transverse to the first bending axis. The cross-section also includes intermediate bending axes between the first and second bending axes that provide resistance to bending that is less than that provided about the first bending axis and greater than that provided about the second bending axis. The connecting elements are positioned along one or more spinal motion segments and engaged to vertebrae with anchors with one of the first, second, and intermediate bending axes in the desired orientation relative to the spinal motion segment to provide the desired stiffness and resistance to bending.

According to one aspect, a system for spinal stabilization comprises an anchor engageable to a vertebral body and a connecting element. The anchor includes a bone engaging portion for engaging the vertebral body and a receiver positionable adjacent the vertebral body. The connecting element includes an elongate body extending along a longitudinal axis between opposite first and second ends. The elongate body is positioned in the receiver of the anchor. The elongate body includes a composite cross-section with a center core comprised of a first material having a first modulus of elasticity and an outer portion around the core comprised of a second material having a second modulus of elasticity that is less than the first modulus elasticity. The cross-section of the core defines a first bending axis orthogonal to the longitudinal axis and a second bending axis transverse to the first bending axis and to the longitudinal axis. The core is stiffer in resistance to bending forces about the first bending axis than about the second bending axis.

According to another aspect, a method for spinal stabilization comprises: engaging an anchor to a vertebral body, wherein the anchor includes a receiver positioned adjacent the first vertebral body; positioning a composite connecting element in the receiver of the anchor, the composite connecting element including an elongated body extending along a longitudinal axis between opposite first and second ends, the composite connecting element including a non-circular core and an outer portion around the core, wherein the core is stiffer than the outer portion and the core defines a number of bending axes orthogonal to the longitudinal axis, the number of bending axes including a first bending axis and a second bending axis transverse to the first bending axis, the core being most stiff in resistance to bending forces about the first bending axis and least stiff to resistance to bending forces about the second bending axis; aligning a selected one of the number of bending axes parallel with the sagittal plane; and locking the composite connecting element in the receiver with the selected bending axis parallel with the sagittal plane.

According to another aspect, a method for stabilizing at least one spinal motion segment comprises: engaging an anchor to a vertebral body; providing a composite connecting element, wherein the connecting element includes an elongated body extending along a longitudinal axis and having a cross-section orthogonal to the longitudinal axis, the cross-section including a non-circular core and a circular outer portion extending around the core, the core being comprised of a material having a higher modulus of elasticity than material comprising the outer portion, the core including a cross-sectional shape defining a number of bending axes each having a different resistance to bending thereabout; selecting one of the number of bending axes and aligning the selected bending axis in a first orientation with the vertebral body; and engaging the connecting element to the anchor with the connecting element positioned in the aligned orientation.

Related features, aspects, embodiments, objects and advantages of the present invention will be apparent from the following description.

### BRIEF DESCRIPTION OF THE DRAWINGS

FIGS. 1A, 1B, and 1C are diagrammatic plan views of a vertebra showing various orientations of a composite connecting element secured to the vertebra with an anchor.

FIGS. 2A and 2B are a perspective view and an end view, respectively, showing the connecting element in FIG. 1A in a minimum stiffness orientation relative to a sagittal plane.

FIGS. 3A and 3B are a perspective view and an end view, respectively, showing the connecting element in FIG. 1B in a maximum stiffness orientation relative to the sagittal plane.

FIGS. 4A and 4B are a perspective view and an end view, respectively, showing the connecting element in FIG. 1C in an intermediate stiffness implantation orientation relative to the sagittal plane.

FIG. 5 is a graph showing the stiffness of the cross-sections of various connecting elements in response to bending forces applied to the connecting elements.

FIGS. 6-10 show end views of various embodiments of composite connecting elements.

### DESCRIPTION OF THE ILLUSTRATED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended. Any such alterations and further modifications in the illustrated devices, and such further applications of the principles of the invention as illustrated herein are contemplated as would normally occur to one skilled in the art to which the invention relates.

A connecting element for connection with anchors engaged to one or more vertebral bodies is provided with a composite cross-section that extends along all or a substantial portion of a length of a body of the connecting element. The composite cross-section provides a maximum resistance to bending in about a first axis and a minimum resistance to bending about a second axis, where the first and second bending axes are generally orthogonal to a longitudinal axis of the connecting element. The connecting element resistance to bending varies between the maximum and minimum resistances when the bending force is applied about intermediate axes that are located between the first and second bending axes. In a preferred embodiment, the composite cross-section is constant in dimension and material properties along the entire length of the connecting element. Other embodiments contemplate cross-sections that vary in dimension and/or material properties along all or a portion of the entire length of the connecting element.

In one example, the connecting element is positioned along the spinal column and engaged to two or more vertebrae of a spinal motion segment so that the first bending axis is oriented in parallel relation to the sagittal plane to provide maximum resistance to extension and flexion motion of the two or more vertebrae of the motion segment. If less resistance to motion in the sagittal plane is desired, the connecting element is rotated about its longitudinal axis so that second bending axis is oriented in parallel relation to the sagittal plane, minimizing resistance to flexion and extension motion of the vertebrae of the motion segment. If the amount of desired or required resistance to flexion and extension motion is greater than that provided about the second bending axis and less than that provided about the first bending axis, then the connecting element can be oriented with an intermediate axis situated between the first and second bending axes aligned in parallel relation to the sagittal plane.

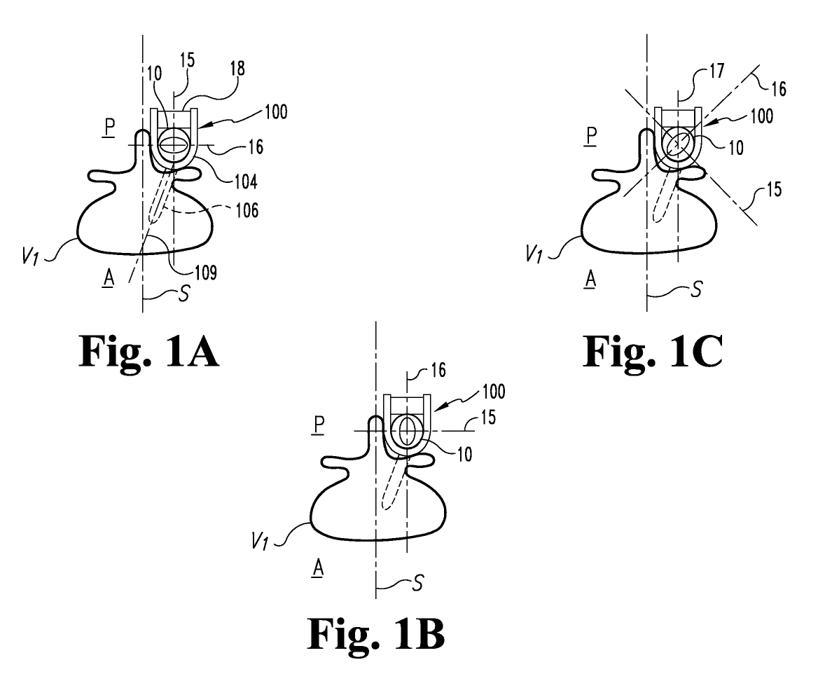

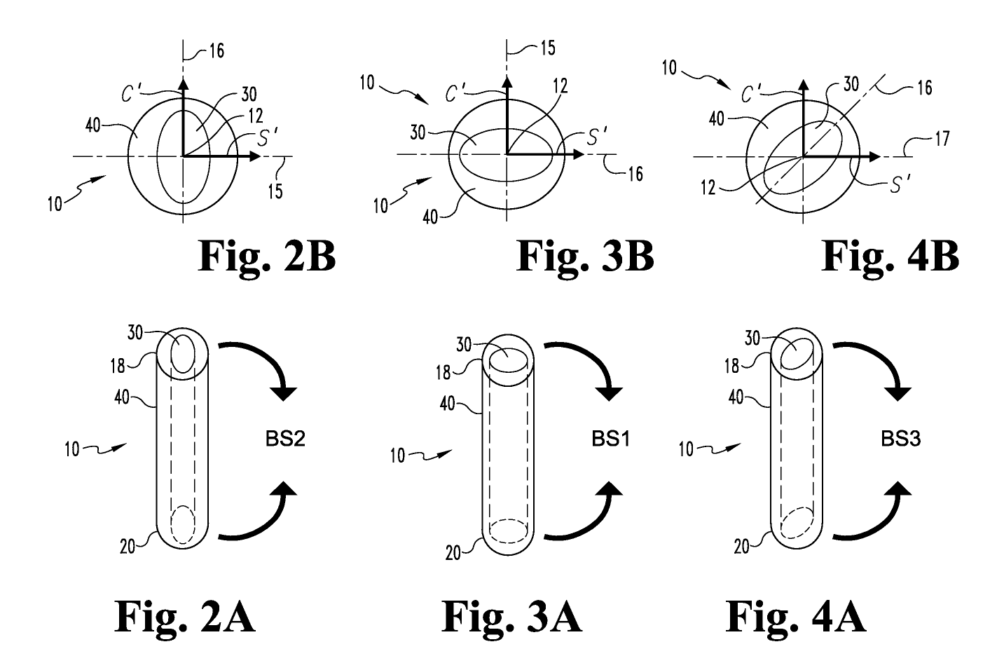

As shown in FIGS. 1A-1C, a connecting element 10 can be secured to the posterior side P of vertebra V1 with one or more anchors 100. As further shown in FIGS. 2A-4B, connecting element 10 is elongated and extends along a central longitudinal axis 12 between opposite first and second ends 18, 20. Connecting element 10 includes a length from first end 18 to second end 20 that is sized to extend along one or more spinal motion segments that include two or more vertebrae and to allow connecting element 10 to be engaged to at least two anchors 100 engaged to respective ones of the at least two vertebrae. Connecting element 10 includes a composite cross-section with a core 30 and an outer portion 40 extending around core 30. Connecting element 10 includes a first bending axis 16 defining a first bending stiffness BS1 thereabout. The cross-section of connecting element 10 also includes a second bending axis 15 transverse to the first bending axis 16 that defines a second bending stiffness BS2 thereabout that differs from the first bending stiffness BS1. In one embodiment, first bending axis 16 is orthogonal to second bending axis 15. However, other embodiments contemplate that bending axes 15, 16 are oblique relative to one another. Furthermore, bending axes 15, 16 can be orthogonal to the central longitudinal axis 12 of connecting element 10. Other embodiments contemplate that one or both of bending axes 15, 16 are oblique to the central longitudinal axis 12 of connecting element 10. In addition, connecting element includes a plurality of intermediate bending axes 17 situated between first bending axis 16 and second bending axis 15. Intermediate bending axes 17 define a third bending stiffness BS3 that is less than first bending stiffness BS1 and greater than second bending stiffness BS2. Connecting element 10 is engaged along the spinal motion segment with one of the bending axes 15, 16, 17 aligned in parallel relation to the sagittal plane S to provide the desired stiffness and resistance to spinal extension and flexion movement of the spinal motion segment.

The composite connecting element 10 includes core 30 preferably made from a material having a higher modulus of elasticity and an outer portion 40 made from a material having a lower modulus of elasticity than the material of core 30. Examples of suitable core material include Grade 5 titanium (Ti-6A1-4V), Commercially Pure Titanium (CP Ti), cobalt-chromium (Co—Cr), stainless steel, Nitinol, and/or carbon-reinforced polyetheretherketone (PEEK). Examples of suitable outer material include those materials with a lower modulus elasticity than that of the selected core material, such as PEEK, polyurethane, epoxy, CP Ti, and/or Nitinol. An outer portion having a greater relative stiffness and a core being more compliant are also contemplated in other embodiments.

Further examples of materials that may be used for the higher modulus core 30 include non-resorbable materials, cobalt-chrome alloys, titanium alloys, superelastic metallic alloys (for example, NITINOL®, GUM METAL®), stainless steel alloys, continuous carbon fiber reinforced PEEK, and/or short carbon fiber reinforced PEEK. Further examples of suitable materials for the lower modulus outer portion include non-resorbable materials, short carbon fiber reinforced PEEK, continuous carbon fiber reinforced PEEK, superelastic metal alloys, polyetherketoneketone (PEKK), polyethylene, and/or polyphenylene.

In one specific example, composite connecting element includes a core and an outer portion that are each comprised of a composite material. For example, in one specific embodiment, core 30 is made of composite material such as 30% short carbon fiber in PEEK, and the outer portion 40 is made of a composite material such as 10% short carbon fiber in PEEK, with a change in carbon fiber content along the radial direction. In another example, the core 30 is 50% continuous or long fiber reinforced PEEK and the outer portion 40 is a thin sleeve made of PEEK.

Another embodiment composite connecting element includes an oval, elliptical, oblong, racetrack, or rectangular core made from Ti-6A1-4V and a circular outer layer around the core made from PEEK. Another example composite connecting element includes an oval, elliptical, oblong, racetrack or rectangular core made from Ti-6A1-4V and a circular outer layer around the core made from polyurethane. Another embodiment composite connecting element includes an oval, elliptical, oblong, racetrack, or rectangular core made from Co—Cr and a circular outer layer around the core made from PEEK. Another example composite connecting element includes an oval, elliptical, oblong, racetrack or rectangular core made from Nitinoland a circular outer layer around the core made from silicone. Another embodiment composite connecting element includes an oval, elliptical, oblong, racetrack, or rectangular core made from stainless steel and a circular outer layer around the core made from epoxy.

Anchor 100 includes a receiver 104 for receiving connecting element 10 therein, and a bone engaging portion 106 for engaging a vertebral body V1. Bone engaging portion 106 can be a threaded screw-like member that extends into and engages the bony structure of vertebral body V1. Other embodiments contemplate that anchor 100 can include a bone engaging portion in the form of a hook, staple, bolt, clamp, cable, or other suitable bone engaging device. Receiver 104 can include a pair of arms defining a passage therebetween for receiving the connecting element 10 therebetween. The arms can be top-loading as shown and internally and/or externally threaded to engage a set screw or other engaging member 108. Other embodiments contemplate receivers that are side-loading, bottom loading, end-loading, clamping members, or any other suitable arrangement for securing connecting element 10 along the spinal column. Receiver 104 can pivot or rotate relative to bone engaging portion 106, or can be fixed relative to the bone engaging portion. In one embodiment, anchor 100 is a bone screw with a U-shaped head pivotally mounted or fixed to the proximal end of a bone screw.

Anchor 100 includes an engagement axis 109 extending toward vertebral body V1 in a generally posterior to anterior direction. Engagement axis 109 is shown obliquely oriented to sagittal plane S, although parallel and orthogonal orientations of engagement axis 109 with sagittal plan S are also contemplated. Bone engaging portion 106 extends in the direction of engagement axis 109 to engage vertebral body V1. Bone engaging portion 106 is shown engaged through the pedicle of vertebral body V1. Other embodiments contemplate bone anchors that are engaged to any other portion of the vertebra, including the spinous process, lamina, transverse processes, or any part or side of the anterior portion A of the vertebra. In addition, connecting element 10 is located in offset relation to sagittal plane S. Other embodiments contemplate connecting element 10 engaged in alignment with sagittal plane S.

Connecting element 10 can be implanted with first bending axis 16, second bending axis 15, or intermediate axis 17 aligned in parallel relation with sagittal plane S. For example, in FIG. 1A connecting element 10 is positioned in receiver 104 of anchor 100, and FIGS. 2A and 2B show connecting element 10 in this orientation in a perspective view and end view, respectively. With second bending axis 15 aligned in a plane S′ that is parallel to sagittal plane S, minimum resistance to bending is provided by connecting element 10 in sagittal plane S. First bending axis 16 is aligned in plane C′ that is in parallel relation with the coronal plane. In FIG. 1B and FIGS. 3A and 3B, connecting element 10 is positioned in anchor 100 with first bending axis 16 aligned in plane S′ that is parallel to sagittal plane S so that maximum resistance to bending is provided in sagittal plane S. Second bending axis 15 is aligned in plane C′ that is in parallel relation with the coronal plane. In FIG. 1C and FIGS. 4A and 4B, connecting element 10 is positioned in anchor 100 with intermediate bending axis 17 aligned in a plane S′ that is parallel to sagittal plane S. In this orientation, the resistance to bending provided in sagittal plane S is less than that provided about axis 16 and greater than that provided about axis 15. First bending axis 16 and second bending axis 15 are aligned in planes that are obliquely oriented to the sagittal and coronal planes of the vertebrae.

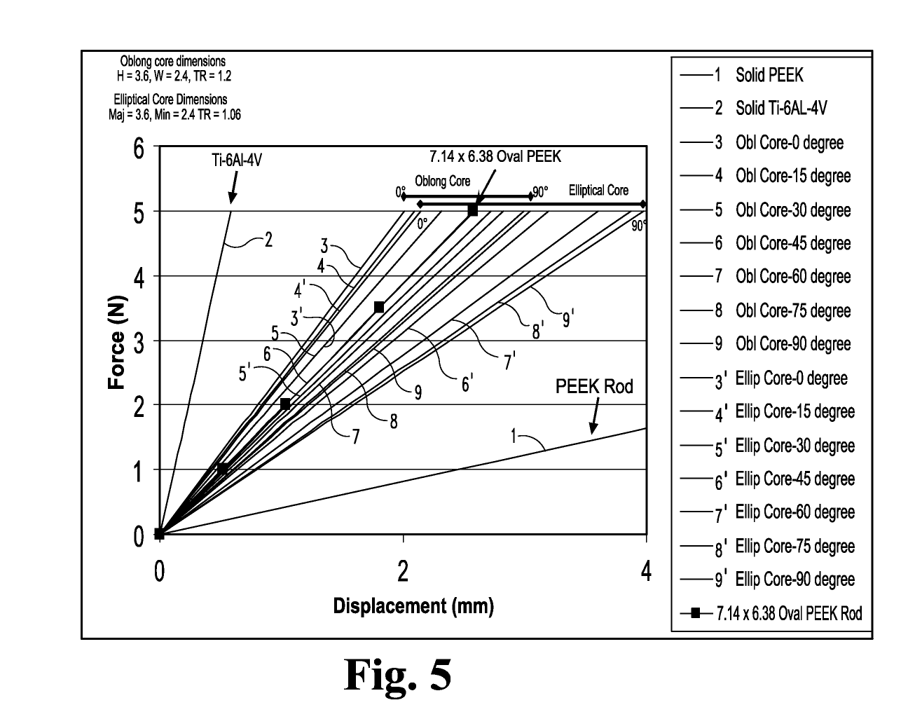

FIG. 5 shows a graph of various embodiment connecting elements and their respective resistance to bending forces and the amount of displacement per unit of applied bending force. The stiffest connecting element tested was a connecting element with a solid, 4.75 millimeter diameter circular cross-section comprised entirely of Ti-6A1-4V as represented by line number 2 in the graph, and the least stiff connecting element tested was a connecting element with a solid, 4.75 millimeter diameter circular cross-section comprised entirely of PEEK material as represented by line number 1 in the graph. In addition, a PEEK connecting element with an oval cross-section comprised of PEEK material and having a major dimension of 7.14 millimeters and minor dimension of 6.38 millimeters was tested with the long dimension of the oval cross-section oriented along the bending axis. Two composite connecting elements each including a stiffer core and less stiff outer portion around the core were also tested. One of the composite connecting elements included an outer portion with a 4.75 millimeter diameter and an oblong core with a height of 3.6 millimeters and a width of 2.4 millimeters. The other composite connecting element included a cross-section having an outer portion with a 4.75 millimeter diameter and an elliptical core having a major dimension of 3.6 millimeters and minor dimension of 2.4 millimeters.

The graph of FIG. 5 shows the bending stiffness of each of the composite connecting elements through various rotational positions of the core relative to the desired bending axis. At the 0 degree orientation, the major dimension of the core is aligned along the bending axis and provides the greatest resistance to bending forces and displacement of the connecting element when subjected to bending forces. At the 90 degree orientation, the minor dimension of the core is aligned along the bending axis and provides the least resistance to bending forces and displacement of the connecting element when subjected to bending forces. The graph also shows the resistance to bending forces at 15 degree incremental rotational positions between the major dimension bending axis and the minor dimension bending axis. Line numbers 3, 4, 5, 6, 7, 8, and 9 show the resistance of the connecting element with the oblong core at the various orientations, and line numbers 3′, 4′, 5′, 6′, 7′, 8′, and 9′ show the resistance of the connecting element with the elliptical core at the various orientations. As shown in the graph, the differing amounts of resistance to bending forces are achieved by rotating the composite connecting elements about their longitudinal axis from the axis of the cross-section aligned with the major dimension of the core to the axis of the cross-section aligned with the minor dimension of the core.

The composite connecting elements tested in FIG. 5 provide a stiffness to resist bending forces and displacement that mimics that provided by the larger cross-section oval PEEK connecting element with a smaller overall size of the cross-section of the composite connecting element. In addition, the composite connecting elements can be rotated about their longitudinal axes between their minimum and maximum bending stiffness to obtain stiffness properties that more closely approximate those provided by the titanium alloy connecting element and the PEEK connecting element, depending on the surgeon preference or desired stabilization characteristic. Thus, various stiffness profiles can be achieved with a single composite connecting element while minimizing or maintaining a smaller cross-section of the connecting element.

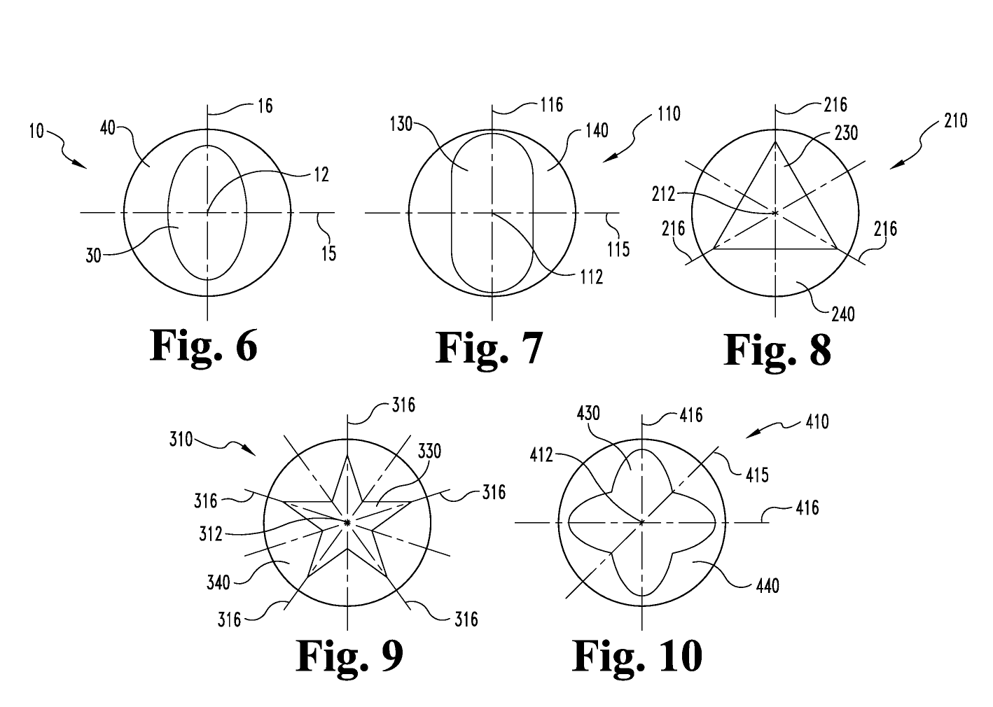

FIGS. 6-10 show various shapes for the cross-sections or ends of various embodiments of the connecting elements discussed herein. In each of FIGS. 6-10, outer portion 40 includes a circular outer shape and provides an isotropic cross-section and therefore is not constrained by the anchor with respect to the rotational positioning of the connecting element about longitudinal axis 12. In FIG. 6, connecting element 10 includes a core 30 with an oval cross-sectional shape that has a major dimension extending along first bending axis 16 and a minor dimension extending along second bending axis 15. Bending axes 15, 16 extend 90 degrees relative to another, allowing the bending resistance of connecting element 10 to be varied between its most stiff and least stiff orientations through a quarter turn of connecting element 10 about longitudinal axis 12.

FIG. 7 shows another embodiment composite connecting element 110 that includes a circular outer portion 140 and a core 130 with an oblong or race-track cross-sectional shape. Core 130 includes rounded ends at its major dimension defining a first bending axis 116, and linear sides extending between the rounded ends that define a minor dimension extending along second bending axis 115. Bending axes 115, 116 extend 90 degrees relative to another, allowing the bending resistance of connecting element 110 to be varied from its most stiff to its least stiff orientation through a quarter turn of connecting element 110 about longitudinal axis 112 to align axis 116 or axis 115 in the direction of bending. Intermediate axes between bending axes 115, 116 can also be aligned in the direction of bending to provide an intermediate bending stiffness.

FIG. 8 shows another embodiment composite connecting element 210 that includes a circular outer portion 240 and a core 230 with a triangular cross-sectional shape. Core 230 includes a major dimension extending through each vertex to the middle of the opposite side of the triangular shape, defining three major bending axes 216. Connecting element 210 provides greatest resistance to bending forces when one of the major bending axes 216 is aligned with the direction of bending. Resistance to bending forces is reduced by rotating connecting element 210 about longitudinal axis 212 to a location aligning an intermediate axis located between major bending axes 216 in the direction of bending.

FIG. 9 shows another embodiment composite connecting element 310 that includes a circular outer portion 340 and a core 330 with a star-shaped cross-section. Core 330 includes a major dimension extending through each vertex of the star, defining five major bending axes 316. Connecting element 310 provides greatest resistance to bending forces when one of the major bending axes 316 is aligned with the direction of bending. Resistance to bending forces is reduced by rotating connecting element 310 about longitudinal axis 312 to a location between major bending axes 316.

FIG. 10 shows another embodiment composite connecting element 410 that includes a circular outer portion 440 and a core 430 with a shape formed by four rounded, interconnected lobes. Core 430 includes rounded ends at the major dimension of opposite ones of the lobes defining two major bending axes 416. The location intermediate the adjacent lobes provides a minor dimension of the core and defines minor bending axes 415. Bending axes 415, 416 extend about 45 degrees relative to another, allowing the bending resistance of connecting element 410 to be varied between its most stiff and least stiff orientations through an eighth turn of connecting element 410 about longitudinal axis 412.

The connecting elements herein include composite cross-sections that allow the surgeon intra-operative freedom to select or adjust the flexion-extension stiffness of the connecting element by selecting the bending axis that is aligned in the direction of bending of the spinal motion segment. The connecting elements preferably include an outer round or circular profile so that the fit between the anchors and the connecting element is not changed or compromised as the connecting element is rotated about its longitudinal axis to select the desired stiffness. The outer surface or ends of the connecting element can be provided with gradations or other marking to assist the surgeon in positioning the connecting element in the desired orientation. The composite connecting elements include a non-circular core that is centrally located in the circular outer portion, although offset locations of the core relative to the outer portion are contemplated. The non-circular cross-section of the core allows the stiffness of the rod in a particular plane of patient motion to be selected or adjusted during implantation or manufacture by changing the orientation of the core relative to the selected plane. In one specific example, the plane of motion of the patient is flexion and extension motion in the sagittal plane of a spinal motion segment including two or more vertebrae.

The surgeon employs the connecting elements discussed herein to vary the stiffness in a plane of motion of the patient by rotating the connecting element about its longitudinal axis to obtain incrementally different stiffness resistance in the plane of motion. The connecting elements include a higher modulus core than the outer portion so that the core contributes a majority of the mechanical properties and stiffness of the connecting element. The connecting elements include an isotropic outer profile so that the orientation of the connecting element does not affect its ability to be engaged to the spinal column using conventional surgical anchors. The connecting elements can be rotated in situ in the patient during the surgical procedure and within a receiver of a bone anchor so that the selected stiffness profile can be secured or locked in position during the procedure.

The core of the connecting elements is preferably made from a higher modulus material and the outer portion is preferable made from a lower modulus material. The connecting element may be linear and straight along its entire length, or may be curved along all or part of its length. The connecting element may be pre-shaped or shaped in the operating room, pre-oriented or oriented in the operating room with the major dimension of the core in a desired orientation. The connecting elements can be manufactured with various manufacturing processes, including over-molding the outer portion on the core, injection molding, extrusion, compression molding, or casting, for example. The core may also include surface treatments, such as shot-peening, grit-blasting, texturing, plasma treatment, anodizing or adhesive, for example, to facilitate and maintain engagement between the outer portion and the core. The composite structures discussed herein also have application with other types of implants, such as screws, plates, or cages.

In certain embodiments, the area of the cross-section and/or the shape of the cross-section of the core of the composite connecting element is constant along the entire length of the connecting element. In other embodiments, the area of the cross-section and/or the shape of the cross-section of the core of the composite connecting element varies along the length of the connecting element. The outer portion surrounding the core may be solid, continuous, non-continuous, braided, knitted, or woven, for example. In addition, the outer portion may be of a composite material or contain any suitable additive.

Although various embodiments have been described as having particular features and/or combinations of components, other embodiments are possible having a combination of any features and/or components from any of embodiments as discussed above. As used in this specification, the singular forms “a,” “an” and “the” include plural referents unless the context clearly dictates otherwise. Thus, for example, the term “a member” is intended to mean a single member or a combination of members, “a material” is intended to mean one or more materials, or a combination thereof. Furthermore, the terms “proximal” and “distal” refer to the direction closer to and away from, respectively, an operator (e.g., surgeon, physician, nurse, technician, etc.) who would insert the medical implant and/or instruments into the patient. For example, the portion of a medical instrument first inserted inside the patient's body would be the distal portion, while the opposite portion of the medical device (e.g., the portion of the medical device closest to the operator) would be the proximal portion.

While the invention has been illustrated and described in detail in the drawings and foregoing description, the same is to be considered as illustrative and not restrictive in character, it being understood that all changes and modifications that come within the spirit of the invention are desired to be protected.

What is claimed is:

1. A system for spinal stabilization, comprising:
an anchor engageable to a vertebral body, wherein said anchor includes a bone engaging portion for engaging the vertebral body and a receiver positionable adjacent the vertebral body; and
a connecting element including an elongate body extending along a longitudinal axis between opposite first and second ends, said elongate body being positioned in and rotatable about said longitudinal axis in said receiver of said anchor, wherein said elongate body includes a composite cross-section with a center core comprised of a first material having a first modulus of elasticity and an outer portion around said core comprised of a second material having a second modulus of elasticity that is less than said first modulus elasticity, said cross-section of said core defining a first bending axis orthogonal to said longitudinal axis and a second bending axis transverse to said first bending axis and to said longitudinal axis, said core being stiffer in resistance to bending forces about said first bending axis than about said second bending axis and said connecting element is rotated in said receiver to align one of said first and second bending axes with a plane of motion of the vertebral body.

2. The system of claim 1, wherein said outer portion includes a circular cross-sectional shape and said core includes a non-circular cross-sectional shape.

3. The system of claim 2, wherein said non-circular cross-sectional shape of said core is oval.

4. The system of claim 2, wherein said non-circular cross-sectional shape of said core is selected from the group consisting of: oval, racetrack, triangular, star and multi-lobed shapes.

5. The system of claim 2, wherein said core consists of metal material and said outer layer consists of polymer material.

6. The system of claim 2, wherein said core consists of a carbon fiber reinforced polymer and said outer portion consists of a polymer.

7. A method for spinal stabilization, comprising:
engaging an anchor to a vertebral body, wherein the anchor includes a receiver positioned adjacent the first vertebral body;
positioning a composite connecting element in the receiver of the anchor, the composite connecting element including an elongated body extending along a longitudinal axis between opposite first and second ends, the composite connecting element including a non-circular core and an outer portion around the core, wherein the core is stiffer than the outer portion and the core defines a number of bending axes orthogonal to the longitudinal axis, the number of bending axes including a first bending axis and a second bending axis transverse to the first bending axis, the core being most stiff in resistance to bending forces about the first bending axis and least stiff to resistance to bending forces about the second bending axis;
aligning a selected one of the number of bending axes parallel with the sagittal plane; and
locking the composite connecting element in the receiver with the selected bending axis parallel with the sagittal plane.

8. The method of claim 7, wherein the core of the composite connecting element is comprised of a first material having a first modulus of elasticity and the outer portion around the core is comprised of a second material having a second modulus of elasticity that is less than the first modulus elasticity.

9. The method of claim 7, wherein the selected bending axis is located between the first bending axis and the second bending axis.

10. The method of claim 7, wherein the selected bending axis is one of the first bending axis and the second bending axis.

11. The method of claim 7, wherein aligning the selected one of the number of bending axes includes rotating the composite connecting element about its longitudinal axis while the composition connecting element is positioned in the receiver of the anchor.

12. The method of claim 7, wherein the outer portion includes a circular cross-sectional shape and said core includes an oval cross-sectional shape, and the first bending axis extends through a major dimension of the oval shape and the second bending axis extends through a minor dimension of the oval shape.

13. The method of claim 12, wherein the first bending axis is orthogonal to the second bending axis.

14. The method of claim 7, wherein the core provides a majority of the stiffness of composite connecting element about each of the number of bending axes.

15. A method for stabilizing at least one spinal motion segment, comprising:
engaging an anchor to a vertebral body;
providing a composite connecting element, wherein the connecting element includes an elongated body extending along a longitudinal axis and having a cross-section orthogonal to the longitudinal axis, the cross-section including a non-circular core and a circular outer portion extending around the core, the core being comprised of a material having a higher modulus of elasticity than material comprising the outer portion, the core including a cross-sectional shape defining a number of bending axes each having a different resistance to bending thereabout;
selecting one of the number of bending axes and aligning the selected bending axis in a first orientation with the vertebral body; and
engaging the connecting element to the anchor with the connecting element positioned in the aligned orientation.

16. The method of claim 15, wherein the number of bending axes are orthogonal to the longitudinal axis and the number of bending axes including a first bending axis and a second bending axis transverse to the first bending axis, the core being most stiff in resistance to bending about the first bending axis and least stiff to resistance to bending about the second bending axis.

17. The method of claim 16, wherein the selected bending axis is one of the first bending axis and the second bending axis and aligning the selected one of the number of bending axes includes rotating the connecting element about its longitudinal axis while the connecting element is positioned in the receiver of the anchor.

18. The method of claim 16, wherein the selected bending axis is located between the first bending axis and the second bending axis and aligning the selected one of the number of bending axes includes rotating the connecting element about its longitudinal axis while the connecting element is positioned in the receiver of the anchor.

19. The method of claim 15, wherein:
aligning the selected bending axis includes aligning the selected bending axis parallel with a sagittal plane of the spinal motion segment; and
locking the connecting element in the receiver with the selected bending axis parallel with the sagittal plane.

20. The method of claim 15, wherein the core of the connecting element is comprised of a first material having a first modulus of elasticity and the outer portion around the core is comprised of a second material having a second modulus of elasticity that is less than said first modulus elasticity.
